(19) United States
(12) Patent Application Publication
(10) Pub. No.: US 2013/0345137 A1
(43) Pub. Date: Dec. 26, 2013

(54) METHOD OF INHIBITING ANGIOGENESIS AND FOOD/BEVERAGE COMPOSITION FOR ANGIOGENESIS INHIBITION

(71) Applicants: Hiroko ITOH, (US); POWERFUL HEALTHY FOOD CORPORATION, NAGANO (JP); SUN CHLORELLA CORP., KYOTO (JP)

(72) Inventors: Hitoshi ITO, MIE (JP); Hiroko ITOH, MIE (JP); Masaki FUJISHIMA, KYOTO (JP); Yukari ARAKAWA, KYOTO (JP); Fukuyoshi NAKADA, NAGANO (JP)

(73) Assignees: Hiroko ITOH, MIE (JP); SUN CHLORELLA CORP., KYOTO (JP); Powerful Healthy Food Corporation, NAGANO (JP)

(21) Appl. No.: 13/760,616

(22) Filed: Feb. 6, 2013

(30) Foreign Application Priority Data
Jun. 20, 2012 (JP) 2012-139227

(51) Int. Cl. A61K 38/16

(52) U.S. Cl. CPC A61K 38/168
USPC 514/13.3

(57) ABSTRACT

A method of inhibiting angiogenesis by applying a protein polysaccharide of a (1→6)-β-D-glucan with a glucan:protein ratio of 55:43 [w/w], obtained by extracting with a 5 w % aqueous solution of sodium hydroxide a residue of extraction and removal of water-soluble polysaccharides from the Agaricus blazei Murill (himematsutake) fruiting body, and a food/beverage composition for angiogenesis inhibition containing the protein polysaccharide of (1→6)-β-D-glucan.

### BACKGROUND OF THE INVENTION

1. Field of the Invention

The present invention relates to a method of inhibiting angiogenesis and a food/beverage composition for angiogenesis inhibition.

2. Description of the Prior Art

Suppression of angiogenesis is thought to lead to suppression of tumor or cancer growth or metastasis, chronic inflammation, rheumatoid arthritis, retinopathy, diabetic retinopathy, age-related macular degeneration, and the like.

It has been reported that an anti-angiogenic action is present in food ingredients such as shark gristle, epigallocatechin (EGC) and epigallocatechin gallate (EGCG), which are components of green tea, genistein, which is a kind of soybean isoflavone, Agaricus blazei-derived ergosterol, and pyroglutamic acid.

Regarding the anti-angiogenic actions of substances derived from mushrooms, in addition to the aforementioned action of Agaricus blazei-derived ergosterol (Isolation of an anti-angiogenic substance from Agaricus blazei Murill: Its antitumor and antimetastatic actions; Kimura et al.; Cancer Sci., September 2004, vol. 95, no. 9, 758-764), the actions of Trametes versicolor-derived PSK (Inhibitory effect of PSK on angiogenesis; Wada T, Wakamatsu Y, et al.; Biotherapy 15(3):389-392, 2001) and Sparassis crispa-derived β-1,3-D-glucan have been reported (Anti-angiogenic and Anti-metastatic Effects of β-1,3-D-Glucan Purified from Hanabiratake, Sparassis crispa; Kyosuke Yamamoto, Takashi Kimura et al.; Biol. Pharm. Bull. 32(2) 259-263, 2009).

PS-K is a glycoprotein containing about 15% of protein, and comprising 19 amino acids such as aspartic acid and glutamic acid, characterized by a putative structure of a main chain of α- or β-1,4 bonds with 1 branch per 5 glucose groups at the 3- or 6-position (Host effects of polysaccharides on cancer-bearing animals, with a focus on the action of glycoprotein PS-K from Trametes versicolor; Shigeru Tsukagoshi; Gan To Kagaku Ryoho (Japanese Journal of Cancer and Chemotherapy), vol. 1, no. 2; pp. 251-257, 1974).

Naohito Ohno et al. reported that the antitumor component of Sparassis crispa is 1,3-β-glucan (Antitumor 1,3-β-Glucan from Cultured Fruit Body of Sparassis crispa; Naohito Ohno et al.; Biol. Pharm. Bull. vol. 23, No. 7, 866-872, 2000). Furthermore, Yamamoto et al. reported that the component had a pulmonary metastasis suppressing effect and anti-angiogenic action (Anti-angiogenic and Anti-metastatic Effects of β-1,3-D-Glucan Purified from Hanabiratake, Sparassis crispa; Kyosuke Yamamoto, Takashi Kimura et al.; Biol. Pharm. Bull. 32(2) 259-263, 2009).

The antitumor activity and antitumor immunological action mechanism of a himematsutake-derived (1→6)-β-D-glucan protein complex have already been reported (Inhibitory Action of a (1→6)-β-D-Glucan-Protein Complex (FIII-2-b) Isolated from Agaricus blazei Murill (“Himematsutake”) on Meth A Fibrosarcoma-Bearing Mice and Its Antitumor Mechanism; Hiroko Itoh et al.; Jpn. J. Pharmacol. 66, 265-271, 1994), and a patent application for an antitumor agent was filed as described in JP-A-HEI-2-78630.

This (1→6)-β-D-glucan protein complex (hereinafter also referred to as “the polysaccharide”) is the first substance of its kind obtained from a mushroom, identified as a polysaccharide comprising 50.2% of sugar moiety and 43.3% of protein moiety (Antitumor Activity and Some Properties of Water-insoluble Hetero-glycans from “Himematsutake,” the Fruiting Body of Agaricus blazei Murill; Takashi Mizuno et al.; Agric. Biol. Chem., 54(11), 2897-2905, 1990).

Known β-(1→6)-glucans include pustulan, which is known to occur in the lichen Umbilicaria pustulata; islandic acid (a polysaccharide comprising a main chain of β-(1→6) bond and a few side chains of 1→4 bond), which is obtained from a culture filtrate of Penicillium islandicum; commercially available dextrans with various molecular weights [Dextran] (produced by Pharmacia Co., a glucan comprising a main chain of α-(1→6) bond and a few branched structures); and oat lichenin, which is isolated from the seeds of oat; however, none of these substances possesses antitumor activity against mouse sarcoma 180 or anti-angiogenic action (<Proceedings of Basic Cancer Study Group Symposium> “Criticism on the anticancer effects of polysaccharides”: Study of antineoplastic polysaccharides in lichen, with special reference to Gyrophora esculenta Miyoshi; Yoshihiro Nishikawa; Nippon Rinsho (Japanese Journal of Clinical Medicine), vol. 27, no. 6; pp. 184-188, 1969).

From the above-described findings, it is suggested that the antitumor activities of polysaccharides may be largely influenced by small differences in their molecular structure and stereochemical factors.

While “the polysaccharide” is known to have the properties described above, there has so far been no knowledge that “the polysaccharide” exhibits anti-angiogenic action.

Regarding the safety of himematsutake (the same himematsutake as that from which “the polysaccharide” was isolated [himematsutake having the same gene sequence of the ITS [internal transcribed spacer] of 5.8S rDNA]), himematsutake was verified to be safe in “Safety research with a focus on the mutagenicity of existing natural food additives and the like” (Health Science Special Research Project) by the Head (Makoto Hayashi) of the Division of Genetics and Mutagenesis in the National Institute of Health Sciences et al. Also in a subchronic toxicity study of himematsutake extract in rats, the safety was verified by Yuichi Kuroiwa et al. at the Division of Pathology in the National Institute of Health Sciences (Lack of subchronic toxicity of an aqueous extract of Agaricus blazei Murill in F344 rats; Y. Kuroiwa, et al.; Food and Chemical Toxicology 43 (2005) 1047-1053).

### SUMMARY OF THE INVENTION

The present invention is intended to provide a method of inhibiting angiogenesis and a food/beverage composition for angiogenesis inhibition that can be administered safely and conveniently for a long period.

[1] It has been discovered that angiogenesis can be inhibited by applying a (1→6)-β-D-glucan-containing product.

In one embodiment of the present invention, there is provided a method of inhibiting angiogenesis by applying a (1→6)-β-D-glucan-containing product.
In another embodiment of the present invention, there is provided an anti-angiogenic agent having a (1→6)-β-D-glucan-containing product as an active ingredient.

In another embodiment of the present invention, there is provided a food/beverage composition for angiogenesis inhibition including a (1→6)-β-D-glucan-containing product.

Angiogenesis can be effectively inhibited by applying a method of inhibiting angiogenesis, the anti-angiogenic agent or food/beverage composition for angiogenesis inhibition of the present invention.

[2] The above-described (1→6)-β-D-glucan-containing product can be a (1→6)-β-D-glucan protein polysaccharide.

[3] The (1→6)-β-D-glucan-containing product can also be a protein polysaccharide having the physicochemical properties (a) to (i) shown below.

(a) Elemental analysis:

### C 41.87%

### H 7.15%

### N 6.88%

Sugar content 50.2%
Protein content 43.3%
(b) Molecular weight: 10,000 to 50,000
(c) Specific rotation:
Levorotary under the conditions:

[α]_{D}²⁵=−23.0°

(c=1.0, 5% NaOH)
(d) Infrared absorption spectrum: Shown in FIG. 1.
(e) Nuclear magnetic resonance spectrum: Shown in FIG. 2.
(f) Solubility: Soluble in alkalis, slightly soluble in water or DMSO (dimethylsulfoxide), insoluble in organic solvents.
(g) Color reaction: Positive for phenol sulfuric acid and ninhydrin reaction.
(h) Color: Greyish white.
(i) Amino acid composition (mol %):

### Asp 10.7

### Thr 5.2

### Ser 5.3

### Glu 11.1

### Gly 9.3

### Ala 11.9

### Val 4.9

### Met 1.1

### Ile 3.3

### Leu 10.8

### Tyr 2.4

### Phe 4.5

### Lys 5.3

### His 2.1

### Arg 5.2

### Pro 6.9

### Total 100%

[4] The above-described (1→6)-β-D-glucan-containing product can be one derived from the Agaricus blazei Murill fruiting body.

The (1→6)-β-D-glucan-containing product derived from the Agaricus blazei Murill fruiting body can be a protein polysaccharide of a (1→6)-β-D-glucan with a glucan:protein ratio of 55:43 [w/w], obtainable by extracting with a 5 w % aqueous solution of sodium hydroxide at 30 degrees Celsius a residue of extraction and removal of water-soluble polysaccharides from the Agaricus blazei Murill fruiting body.

[5] In the method of inhibiting angiogenesis of the present invention, (1→6)-β-D-glucan-containing product can be orally administered. The anti-angiogenic agent of the present invention can be a preparation for oral administration.

[6] The method of inhibiting angiogenesis and the anti-angiogenic agent and food/beverage composition for angiogenesis inhibition of the present invention are preferably those that inhibit angiogenesis induced by vascular endothelial growth factor (VEGF).

[7] The method of inhibiting angiogenesis and the anti-angiogenic agent and food/beverage composition for angiogenesis inhibition of the present invention are preferably those that inhibit angiogenesis by reducing the secretion of vascular endothelial growth factor (VEGF).

Herein, “an anti-angiogenic agent” can be reworded to “a substance for anti-angiogenic agents”.

The method of inhibiting angiogenesis by applying the anti-angiogenic agent or food/beverage composition for anti-angiogenic agents of the present invention can be reworded as a method of inhibiting angiogenesis by applying the (1→6)-β-D-glucan-containing product according to [1] to [4] above.

The method of inhibiting angiogenesis of the present invention can be used safely and conveniently for a long period, and is capable of effectively inhibiting angiogenesis. The anti-angiogenic agent and food/beverage composition for angiogenesis inhibition of the present invention can be administered safely and conveniently for a long period, and is capable of effectively inhibiting angiogenesis.

### BRIEF DESCRIPTION OF THE DRAWINGS

FIG. 1 is an infrared absorption spectrum.

FIG. 2 is a nuclear magnetic resonance spectrum.

FIG. 3 is a graphic representation showing the influence of (1→6)-β-D-glucan protein polysaccharide on tumor-induced vascularization as determined by the dorsal air sac assay.

FIG. 4 is a graphic representation showing the influence of (1→6)-β-D-glucan protein polysaccharide on VEGF-induced angiogenesis as determined by Matrigel plug assay.

FIG. 5 is a graphic representation showing the human umbilical venous endothelial cell proliferation inhibitory effect of (1→6)-β-D-glucan protein polysaccharide.

### DETAILED DESCRIPTION OF THE INVENTION

[1] In the method of inhibiting angiogenesis of the present invention, a (1→6)-β-D-glucan-containing product such as a (1→6)-β-D-glucan protein polysaccharide is applied. The active ingredient of the anti-angiogenic agent in the present invention is a (1→6)-β-D-glucan-containing product such as a (1→6)-β-D-glucan protein polysaccharide.

(1) The above mentioned (1→6)-β-D-glucan-containing product as an active ingredient of the anti-angiogenic agent can, for example, be obtained as described below.

The fresh fruiting body or dried fruiting body of Agaricus blazei Murill (himematsutake) is used as shredded for the fresh fruiting body, or as milled for the dry product. The shredded or milled fruiting body is subjected to a pre-extraction treatment with a lower aliphatic alcohol or a lower aliphatic alcohol containing not more than 20% of water to remove low-molecular components.

Next, the residue after extraction is subjected to hot water extraction, and the water-soluble components are removed. This extraction residue is air-dried and subjected to extraction with an aqueous solution of ammonium oxalate. Furthermore, the extraction residue is subjected to extraction at 30 degrees Celsius with the addition of a 5% aqueous solution of sodium hydroxide.

This extract is neutralized with acetic acid and adjusted to a pH of 5 to 6, and the resulting precipitate is removed. Next, the protein polysaccharide component is separated by gel filtration. Furthermore, this was dialyzed and purified by desalinization by another publicly known means of purification; after the purified liquid is concentrated, the concentrate is lyophilized by a conventional method to yield a greyish white powder.

(2) Physicochemical Properties

The physicochemical properties of the above mentioned (1→6)-β-D-glucan-containing product such as a (1→6)-β-D-glucan protein polysaccharide as an active ingredient of the anti-angiogenic agent are described below.

(a) Elemental analysis results

C, 41.8%; H, 7.15%; N, 6.88%; a protein polysaccharide having a sugar content (based on glucose) of 43.3% as determined by the phenol sulfuric acid method and a protein content (based on bovine albumin) of 50.2% as determined by the Lowry method.

The sugar composition mostly comprises glucose as determined by alditol acetate gas chromatography, and contains trace amounts of xylose, galactose, and maltose. Methylation analysis by the Hakomori method and 1H- and 13C-NMR analysis reveal β1-6 bonds of glucose.

(b) The molecular weight as determined by gel filtration is about 10,000 to 50,000.
(c) Optical rotation:
Levorotary under the conditions:

[α]_{D}²⁵=−23.0°

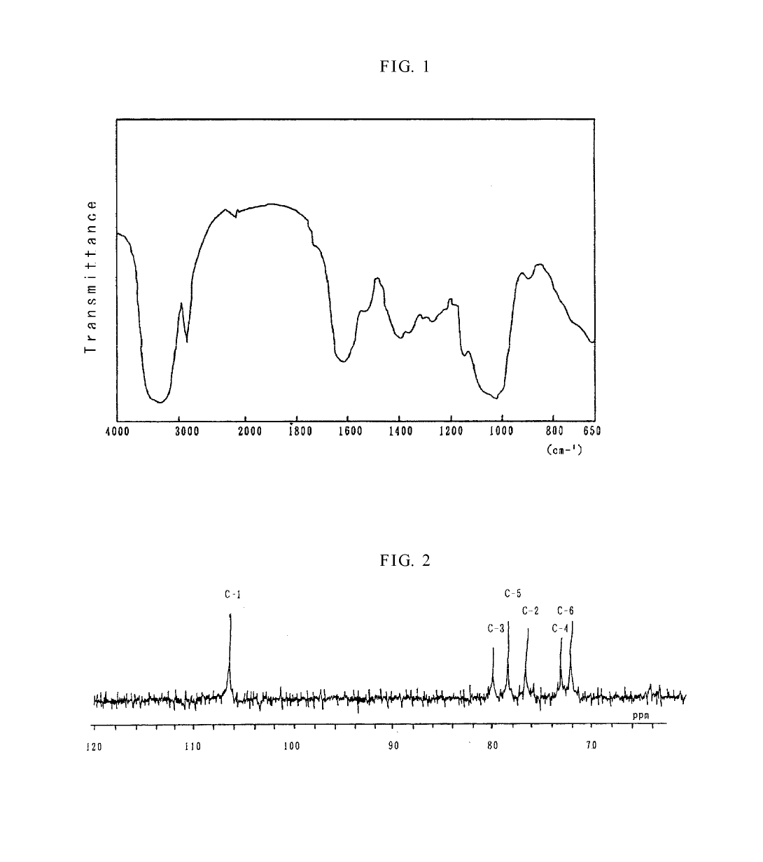

(c=1.0, 5% NaOH)
(d) Results of a determination of the infrared absorption spectrum by the KBr disc method are shown in FIG. 1.
(e) Results of a determination of the nuclear magnetic resonance spectrum (13C-NMR) in solution in 0.3M NaOD are shown in FIG. 2.
(f) Soluble in alkalis such as 1 to 10% sodium hydroxide, slightly soluble in water, acids, and DMSO (dimethylsulfoxide), and insoluble in organic solvents, for example, ethanol, acetone, ether, and chloroform.
(g) Positive for the anthrone sulfuric acid reagent and the ninhydrin reaction reagent.
(h) The powder obtained by lyophilization has a greyish white color.
(i) The amino acid composition (mol %)
The product hydrolyzed with 6M hydrochloric acid at 110 degrees Celsius for 20 hours is determined using the Hitachi 835 model amino acid analyzer; the results are Asp 10.7, Thr 5.2, Ser 5.3, Glu 11.1, Gly 9.3, Ala 11.9, Val 4.9, Met 1.1, Ile 3.3, Leu 10.8, Tyr 2.4, Phe 4.5, Lys 5.3, His 2.1, Arg 5.2, Pro 6.9.

(3) Acute Toxicity

In an acute toxicity study in ICR mice (female, 5 weeks of age) by a single intraperitoneal or oral dose (1-week observation), no remarkable body weight change was observed in the mice receiving 500 mg/kg of the above-described (1→6)-β-D-glucan-containing product in the present invention with a mortality rate of 0/7 for both routes of administration (JP-A-HEI-2-78630).

[2] The (1→6)-β-D-glucan-containing product such as the above-described (1→6)-β-D-glucan protein polysaccharide applied in the method of inhibiting angiogenesis of the present invention or an active ingredient of the anti-angiogenic agent of the present invention is suitable for oral administration; its adult dose is suitably about 0.1 to 10 g per day. However, this is not to be construed as limiting, because (1→6)-β-D-glucan-containing products such as (1→6)-β-D-glucan protein polysaccharidees are not toxic to the human body.

Said (1→6)-β-D-glucan-containing product is not subject to limitations with respect to the dosage form for oral administration, and can, for example, be formulated in powders, tablets, hard capsules, and soft capsules.

In forming various dosage forms, various excipients, binders, disintegrants, lubricants, coating agents, coloring agents, taste correctives, odor correctives, plasticizers, and the like can be used as appropriate.

Examples of excipients include sugars (lactose, sucrose, glucose, mannitol), starches (potato, wheat, corn), inorganic substances (calcium carbonate, calcium sulfate, sodium hydrogen carbonate, sodium chloride), crystalline cellulose, plant powders (licorice powder, gentian powder), and the like.

Examples of binders include starch paste liquid, gum arabic, gelatin, sodium alginate, methylcellulose (MC), ethylcellulose (EC), polyvinylpyrrolidone (PVP), polyvinyl alcohol (PVA), hydroxypropylcellulose (HPC), carboxymethylcellulose (CMC), and the like.

Examples of disintegrants include starches, agar, gelatin powder, crystalline cellulose, CMC-Na, CMC-Ca, calcium carbonate, sodium hydrogen carbonate, sodium alginate, and the like.

Examples of lubricants include magnesium stearate, talc, hydrogenated vegetable oils, macrogol, silicone oil, and the like.

Examples of coating agents include sugar coatings (sucrose, HPC, shellac), glue coatings (gelatin, glycerine, sorbitol), film coatings [hydroxypropylmethylcellulose (HPMC), EC, HPC, PVP], enteric coatings [hydroxypropylmethylcellulose phthalate (HPMCP), cellulose acetate phthalate (CAP)], and the like.

Examples of coloring agents include water-soluble food dyes, lake pigments, and the like. Examples of taste correctives include lactose, sucrose, glucose, mannitol, and the like. Examples of odor correctives include aromatic essential oils), light screens (titanium oxide) and the like. Examples of plasticizers include phthalic acid esters, vegetable oils, polyethylene glycol, and the like.

The food/beverage composition for angiogenesis inhibition of the present invention can be in, for example, a form containing a (1→6)-β-D-glucan-containing product such as a (1→6)-β-D-glucan protein polysaccharide in a food/beverage composition that constitutes a nutritional food, nutritional supplement, beverage, or the like, and may comprise various components that do not interfere with the effect of the present invention.

Such method of inhibiting angiogenesis of the present invention can be used safely and conveniently for a long period, and is capable of effectively inhibiting angiogenesis. Such anti-angiogenic agents and food/beverage compositions for anti-angiogenic agents can be administered safely and conveniently for a long period, and are capable of effectively inhibiting angiogenesis.

### EXAMPLES

A (1→6)-β-D-glucan protein polysaccharide [hereinafter also referred to as “ABP”] was tested for anti-angiogenic effects in oral administration.

(1) Test Substance and Test Animals

According to the procedure of Kawagishi et al. [Kawagishi H, Kanao T, et al.: Formolysis of a potent antitumor (1-6)-β-D-glucan-protein complex from Agaricus blazei fruiting bodies and antitumor activity of the resulting products. Carbohydr Polym 12: 393-403 1990], after extraction of a hot-water-soluble polysaccharide from the fruiting bodies of Agaricus blazei Murill (himematsutake), water-insoluble polysaccharides were obtained by successive extraction with 1% ammonium oxalated solution, 5% sodium hydroxide solution, 20% sodium hydroxide solution, and 5% lithium chloride-dimethylacetamide solution in that order. These water-insoluble fractions were further fractionated by ethanol precipitation, gel-filtration, and the like to yield the test substance (1→6)-β-D-glucan protein polysaccharide. Its physicochemical properties are as shown above.

Five-week-old female A/J Jms Slc mice weighing 15-20 g (obtained from Japan SLC, Inc.) were used as test animals. These mice maintained on a CE-7 pellet diet (produced by CLEA Japan, Inc.) with free access to water.

(2) Measurement of Tumor-Induced Angiogenesis in the Dorsal Air Sac (DAS) Assay

DAS assay was carried out according to the method described by Yamamoto et al. [Yamamoto K, Kimura T: Anti-angiogenic and anti-metastatic effects of β-1,3-D-glucan purified from Hanabiratake, Sparassis crispa. Biol Pharm Bull 32(2): 259-263 2009] with a slight modification.

Specifically, both sides of a diffusion chamber ring were covered with membrane filters; the resulting chambers were filled with 5×10⁵ cells of P-7423 cells (lung tumor cells 7423) in 150 μL of PBS. Each P-7423 containing chamber was implanted into the DAS of female A/J Jms Slc mice on day 0. The control group was implanted with PBS containing chamber. In all cases, the PBS was an antibacterial/antifungal solution containing 10,000 U/mL penicillin in phosphate-buffered physiological saline (×100) (produced by Wako Pure Chemical Industries).

ABP was orally administered twice a day from day 1 to day 14.

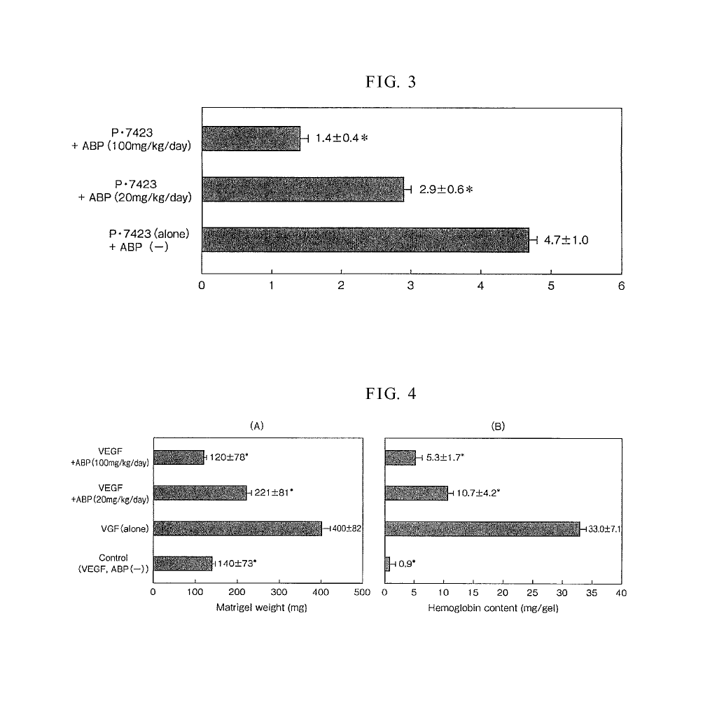

On day 15, each mouse was sacrificed and tumor cell-induced angiogenesis at the implanted zone was observed. FIG. 3 shows the number of newly formed blood vessels longer than 3 mm in length with a characteristic zigzag shape of newly formed blood vessels in the chamber implanted zone. In FIG. 3, values are indicated as the mean±standard error (n=7); * indicates a statistically significant difference (p<0.05, versus P-7423-alone control group).

Angiogenesis was strongly induced after implantation of P-7423 cells in the chamber; however, oral administration of ABP significantly inhibited the tumor-induced angiogenesis.

(3) Measurement of VEGF-Induced Angiogenesis in the Matrigel Plug Assay

The Matrigel plug assay was performed according to the method described by Passaniti et al. [Passaniti A, Taylor R M, et al.: A simple, quantitative method for assessing angiogenesis and antiangiogenic agents using reconstituted basement membrane, heparin, and fibroblast growth factor. Lab Invest 67: 519-528 1992].

Specifically, each female A/J Jms Slc mice was subcutaneously injected with 0.5 mL of Matrigel containing 20 ng/mL of VEGF (recombinant mouse VEGF, obtained from Sigma Co., [USA]) and 32 U/mL heparin on day 0. The control group was injected with Matrigel alone.

ABP was orally administered twice a day from day 1 to day 14.

On day 15, each Matrigel was excised and weighed, and then the gel was treated with dispase-II (1.5 mg/mL, produced by Roche Diagnostics Co.), followed by determination of hemoglobin content using the Quantichrom Hemoglobin assay kit (produced by FUNAKOSHI Co., Ltd.). The results are shown in FIG. 4.

In FIG. 4, (A) shows Matrigel weight, and (B) shows the hemoglobin content in Matrigel. In FIG. 4, values are indicated as the mean±standard error (n=7); * indicates a statistically significant difference (p<0.05, versus VEGF-alone control group).

Remarkable increase in the Matrigel weight and the hemoglobin content was observed in the group injected with Matrigel containing VEGF compared to the group treated with Matrigel alone. Conversely, such angiogenic responses were significantly suppressed by oral administration of ABP.

(4) VEGF Secretion in P-7423 (Lung Tumor Cells 7423) Cells

P-7423 lung carcinoma cells were grown in 24 well plates in complete medium and incubated at 37 degrees Celsius, 5% gaseous carbon dioxide for 24 hours).

The medium was replaced with RPMI-1640 (a single-cell suspension of 5×10⁵ viable cells of P-7423 line) with FBS (fetal bovine serum, obtained from Gibco BRL Co. [New Zealand]) and the 0, 20, 200 μg/mL ABP, and incubated at 37 degrees Celsius, 95% nitrogen/5% gaseous carbon dioxide for 18 hours in hypoxia, after which conditioned media were analyzed for VEGF protein content by an ELISA kit (produced by Santa Cruz Biotechnology, Inc.).

Table 1 shows the effect of ABP on VEGF expression in culture supernatants of P-7423 cell in hypoxia as VEGF (pg/mL) of P-7423 cells at each ABP concentration. In Table 1, each value is shown as the mean±standard error (n=4); * indicates a statistically significant difference (p<0.01, versus control group).

TABLE 1

| ABP dose (μg/ml) | VEGF (pg/ml) of P · 7423 in hypoxia |
|---|---|
| 0 | 262.5 ± 22.4 |
| 20 | 259.7 ± 30.1 |
| 200 | 228.0 ± 20.5* |

As shown in Table 1, 200 μg/mL ABP decreased the secreted VEGF content significantly.

(5) HUVEC Proliferation Assays

Angiogenesis depends on several aspects such that the endothelial cells must proliferate to provide the necessary number of cells for the growing vessels, and the cells need to be capable of migration, etc. Due to the key role vascular endothelial cell plays in angiogenesis, the effect of ABP on HUVEC was explored.

HUVEC (human umbilical venous endothelial cells, obtained from Kurabo Industries Co.) were suspended in a DMEM (Dulbecco's modified Eagle medium, obtained from Nissui Pharmaceutical Co.) with 2% (v/v) FBS (fetal bovine serum, obtained from Gibco BRL Co. [New Zealand]) (40,000 cells/mL), plated onto gelatinized 96-well culture plates (0.1 mL/well), and incubated (37 degrees Celsius, 5% gaseous carbon dioxide for 24 hours).

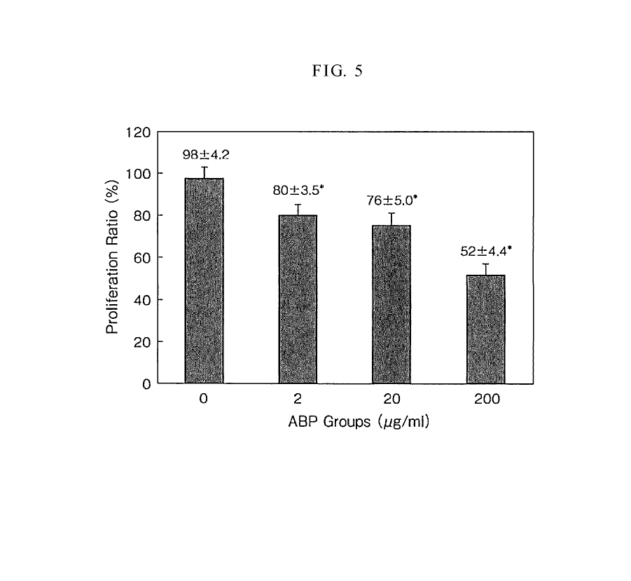

The medium were replaced with 0.1 mL of the DMEM with 5% (v/v) FBS, and incubated for 24, 48, and 72 hours, or with the addition of 2, 20, 200 μg/mL ABP (for 48 hours, 37 degrees Celsius, 5% gaseous carbon dioxide). Cell proliferation was determined using MTT assay (Carmichael J, DeGraff W G, et al.: Evaluation of a tetrazolium-based semiautomated colorimetric assay. Assessment of chemosensitivity testing. Cancer Res 47: 936-942 1987). The results are shown in FIG. 5.

In FIG. 5, the proliferation ratio (%) indicates the cell proliferation rate in different dose ABP medium compared with DMEM when cell numbers are considered as 100%. In FIG. 5, each value is shown as the mean±standard error (n=6); * indicates a statistically significant difference (p<0.05, versus DMEM-treated control group).

ABP at 2, 20, 200 μg/mL inhibited the proliferation of HUVEC in a dose-dependent fashion.

Specifically, ABP was shown to directly inhibit HUVEC cell proliferation in vitro. However the mechanism of ABP inhibitory effect on HUVEC is unclear, which could be the cytotoxicity effect.

It has already been found that tumor-induced neovascularization is inhibited by ergosterol isolated from Agaricus blazei. Fungal protein-bound polysaccharide (Krestin) derived from C. vesicolor was also reported to act as an anti-angiogenic agent.

However, Krestin is mainly constituted of β-1,4-bond glucan main chain having β-1,3 and β-1,6 bond side chain binding to a protein moiety.

Therefore, it is considered that ABP having (1→6)-β-D-glucan is a novel anti-angiogenic agent being particularly effective in oral administration.

1-15. (canceled)

16. The food/beverage composition for angiogenesis inhibition comprising a (1→6)-β-D-glucan-containing product, wherein:
the (1→6)-β-D-glucan-containing product is a protein polysaccharide of a (1→6)-β-D-glucan, which has a sugar content based on glucose of 43.3% as determined by the phenol sulfuric acid method and a protein content based on bovine albumin of 50.2% as determined by the Lowry method, obtained by the steps of:
extracting a fruiting body of Agaricus blazei Murill with a lower aliphatic alcohol or a lower aliphatic alcohol containing not more than 20% of water to remove low-molecular components as a pre-extraction treatment;
extracting the residue of the extraction as a pre-extraction treatment with hot water to remove water-soluble components;
air-drying the residue of the hot water extraction;
extracting the air-dried hot water extraction residue with an aqueous solution of ammonium oxalate;
subjecting the residue of the ammonium oxalate solution extraction to extraction at 30 degrees Celsius with the addition of a 5% aqueous solution of sodium hydroxide;
neutralizing the extract of the sodium hydroxide solution extraction with acetic acid;
adjusting the pH of the extract to 5 to 6 and removing the resulting precipitate;
separating the protein polysaccharide component by gel filtration; and
dialyzing the separated protein polysaccharide component and purifying the protein polysaccharide by desalinization.

17. The food/beverage composition for angiogenesis inhibition according to claim 16, wherein:
the composition directly suppresses the growth of venous endothelial cells.

18. The food/beverage composition for angiogenesis inhibition comprising a (1→6)-β-D-glucan-containing product, wherein:
the (1→6)-β-D-glucan-containing product is a protein polysaccharide of a (1→6)-β-D-glucan, which has a sugar content based on glucose of 43.3% as determined by the phenol sulfuric acid method and a protein content based on bovine albumin of 50.2% as determined by the Lowry method, obtained by the steps of:
extracting a fruiting body of Agaricus blazei Murill to extract a hot-water-soluble polysaccharide; and
successively extract the residue of the extraction with 1% ammonium oxalated solution, 5% sodium hydroxide solution, 20% sodium hydroxide solution, and 5% lithium chloride-dimethylacetamide solution in that order to obtain water-insoluble polysaccharides,
further fractionating thus obtained water-insoluble polysaccharides.

19. The food/beverage composition for angiogenesis inhibition according to claim 18, wherein the composition directly suppresses the growth of venous endothelial cells.

20. The food/beverage composition for angiogenesis inhibition comprising a (1→6)-β-D-glucan-containing product, wherein:
the (1→6)-β-D-glucan-containing product is a protein polysaccharide having the physicochemical properties (a) to (h) shown below,
(a) Elemental analysis:
C 41.87%
H 7.15%
N 6.88%
Sugar content 50.2%
Protein content 43.3%
(b) Molecular weight: 10,000 to 50,000
(c) Specific rotation:
[α]_{D}^{25≡}−23.0°
(c=1.0, 5% NaOH)
(d) Infrared absorption spectrum: Shown in FIG. 1,
(e) Nuclear magnetic resonance spectrum: Shown in FIG. 2,
(f) Solubility: Soluble in alkalis, and insoluble in organic solvents,
(g) Color reaction: Positive for phenol sulfuric acid and ninhydrin reaction,
(h) Amino acid composition (mol %):
Asp 10.7
Thr 5.2
Ser 5.3
Glu 11.1
Gly 9.3
Ala 11.9
Val 4.9
Met 1.1
Ile 3.3
Leu 10.8
Tyr 2.4
Phe 4.5
Lys 5.3
His 2.1
Arg 5.2
Pro 6.9
Total 100%; and
the composition inhibits angiogenesis induced by vascular endothelial growth factor.

21. The food/beverage composition for angiogenesis inhibition according to claim 20, wherein the composition inhibits angiogenesis by reducing the secretion of vascular endothelial growth factor.

22. The food/beverage composition for angiogenesis inhibition comprising a (1→6)-β-D-glucan-containing product, wherein the (1→6)-β-D-glucan-containing product is a protein polysaccharide having the physicochemical properties (a) to (h) shown below,
(a) Elemental analysis:
C 41.87%
H 7.15%
N 6.88%
Sugar content 50.2%
Protein content 43.3%
(b) Molecular weight: 10,000 to 50,000
(c) Specific rotation:
[α]_{D}^{25≡}−23.0°
(c=1.0, 5% NaOH)
(d) Infrared absorption spectrum: Shown in FIG. 1.
(e) Nuclear magnetic resonance spectrum: Shown in FIG. 2.
(f) Solubility: Soluble in alkalis, and insoluble in organic solvents.
(g) Color reaction: Positive for phenol sulfuric acid and ninhydrin reaction.
(h) Amino acid composition (mol %):
Asp 10.7
Thr 5.2
Ser 5.3
Glu 11.1
Gly 9.3
Ala 11.9
Val 4.9
Met 1.1
Ile 3.3
Leu 10.8
Tyr 2.4
Phe 4.5
Lys 5.3
His 2.1
Arg 5.2
Pro 6.9
Total 100%; and
the composition directly suppresses the growth of venous endothelial cells.

23. The food/beverage composition for angiogenesis inhibition according to claim 20, wherein said protein polysaccharide is slightly soluble in water or DMSO (dimethylsulfoxide) and Greyish white in color.

24. The food/beverage composition for angiogenesis inhibition according to claim 23, wherein the composition inhibits angiogenesis by reducing the secretion of vascular endothelial growth factor.

25. The food/beverage composition for angiogenesis inhibition according to claim 22, wherein said protein polysaccharide is slightly soluble in water or DMSO (dimethylsulfoxide) and Greyish white in color.

26. A method of inhibiting angiogenesis by applying a (1→6)-β-D-glucan-containing product, wherein:
the (1→6)-β-D-glucan-containing product is a protein polysaccharide of a (1→6)-β-D-glucan, which has a sugar content based on glucose of 43.3% as determined by the phenol sulfuric acid method and a protein content based on bovine albumin of 50.2% as determined by the Lowry method, obtained by the steps of:
extracting a fruiting body of Agaricus blazei Murill with a lower aliphatic alcohol or a lower aliphatic alcohol containing not more than 20% of water to remove low-molecular components as a pre-extraction treatment;
extracting the residue of the extraction as a pre-extraction treatment with hot water to remove water-soluble components;
air-drying the residue of the hot water extraction;
extracting the air-dried hot water extraction residue with an aqueous solution of ammonium oxalate;
subjecting the residue of the ammonium oxalate solution extraction to extraction at 30 degrees Celsius with the addition of a 5% aqueous solution of sodium hydroxide;
neutralizing the extract of the sodium hydroxide solution extraction with acetic acid;
adjusting the pH of the extract to 5 to 6 and removing the resulting precipitate;
separating the protein polysaccharide component by gel filtration; and
dialyzing the separated protein polysaccharide component and purifying the protein polysaccharide by desalinization.

27. The method of inhibiting angiogenesis according to claim 26 wherein said protein polysaccharide directly suppresses the growth of venous endothelial cells.

28. A method of inhibiting angiogenesis by applying a (1→6)-β-D-glucan-containing product, wherein:
the (1→6)-β-D-glucan-containing product is a protein polysaccharide of a (1→6)-β-D-glucan, which has a sugar content based on glucose of 43.3% as determined by the phenol sulfuric acid method and a protein content based on bovine albumin of 50.2% as determined by the Lowry method, obtained by the steps of:
extracting a fruiting body of Agaricus blazei Murill to extract a hot-water-soluble polysaccharide; and
successively extract the residue of the extraction with 1% ammonium oxalated solution, 5% sodium hydroxide solution, 20% sodium hydroxide solution, and 5% lithium chloride-dimethylacetamide solution in that order to obtain water-insoluble polysaccharides, further fractionating thus obtained water-insoluble polysaccharides.

29. The method of inhibiting angiogenesis according to claim 28, wherein said protein polysaccharide directly suppresses the growth of venous endothelial cells.

30. A method of inhibiting angiogenesis by applying a (1→6)-β-D-glucan-containing product, wherein:
the (1→6)-β-D-glucan-containing product is a protein polysaccharide having the physicochemical properties (a) to (h) shown below,
(a) Elemental analysis:
C 41.87%
H 7.15%
N 6.88%
Sugar content 50.2%
Protein content 43.3%
(b) Molecular weight: 10,000 to 50,000
(c) Specific rotation:
[α]_{D}^{25≡}−23.0°
(c=1.0, 5% NaOH)
(d) Infrared absorption spectrum: Shown in FIG. 1.
(e) Nuclear magnetic resonance spectrum: Shown in FIG. 2.
(f) Solubility: Soluble in alkalis, and insoluble in organic solvents.
(g) Color reaction: Positive for phenol sulfuric acid and ninhydrin reaction.
(h) Amino acid composition (mol %):
Asp 10.7
Thr 5.2
Ser 5.3
Glu 11.1
Gly 9.3
Ala 11.9
Val 4.9
Met 1.1
Ile 3.3
Leu 10.8
Tyr 2.4
Phe 4.5
Lys 5.3
His 2.1
Arg 5.2
Pro 6.9
Total 100%; and
said protein polysaccharide inhibits angiogenesis induced by vascular endothelial growth factor.

31. The method of inhibiting angiogenesis according to claim 30, wherein the product is applied orally.

32. The method of inhibiting angiogenesis according to claim 30, wherein said protein polysaccharide inhibits angiogenesis by reducing the secretion of vascular endothelial growth factor.

33. The method of inhibiting angiogenesis according to claim 32, wherein the product is applied orally.

34. A method of inhibiting angiogenesis by applying a (1→6)-β-D-glucan-containing product, wherein:
the (1→6)-β-D-glucan-containing product is a protein polysaccharide having the physicochemical properties (a) to (h) shown below,
(a) Elemental analysis:
C 41.87%
H 7.15%
N 6.88%
Sugar content 50.2%
Protein content 43.3%
(b) Molecular weight: 10,000 to 50,000
(c) Specific rotation:
[α]_{D}^{25≡}−23.0°
(c=1.0, 5% NaOH)
(d) Infrared absorption spectrum: Shown in FIG. 1.
(e) Nuclear magnetic resonance spectrum: Shown in FIG. 2.
(f) Solubility: Soluble in alkalis, and insoluble in organic solvents.
(g) Color reaction: Positive for phenol sulfuric acid and ninhydrin reaction.
(h) Amino acid composition (mol %):
Asp 10.7
Thr 5.2
Ser 5.3
Glu 11.1
Gly 9.3
Ala 11.9
Val 4.9
Met 1.1
Ile 3.3
Leu 10.8
Tyr 2.4
Phe 4.5
Lys 5.3
His 2.1
Arg 5.2
Pro 6.9
Total 100%; and
said protein polysaccharide directly suppresses the growth of venous endothelial cells.

35. The method of inhibiting angiogenesis according to claim 34, wherein the product is applied orally.

36. The method of inhibiting angiogenesis according to claim 30, wherein said protein polysaccharide is slightly soluble in water or DMSO (dimethylsulfoxide) and Greyish white in color.

37. The method of inhibiting angiogenesis according to claim 36, wherein the product is applied orally.

38. The method of inhibiting angiogenesis according to claim 36, wherein said protein polysaccharide inhibits angiogenesis by reducing the secretion of vascular endothelial growth factor.

39. The method of inhibiting angiogenesis according to claim 38 wherein the product is applied orally.

40. The method of inhibiting angiogenesis according to claim 34 wherein said protein polysaccharide is slightly soluble in water or DMSO (dimethylsulfoxide) and Greyish white in color.

41. The method of inhibiting angiogenesis according to claim 40 wherein the product is applied orally.

42. A composition obtained by the steps of:
extracting a fruiting body of Agaricus blazei Murill with a lower aliphatic alcohol or a lower aliphatic alcohol containing not more than 20% of water to remove low-molecular components and to produce a residue;
treating the residue of the extraction with hot water to remove water-soluble components;
air-drying a residue of the hot water treatment;
extracting the air-dried hot water treatment residue with an aqueous solution of ammonium oxalate to produce a residue;
subjecting the residue of the ammonium oxalate solution extraction to extraction at 30 degrees Celsius with the addition of a 5% aqueous solution of sodium hydroxide to produce an extract;
neutralizing the extract of the sodium hydroxide solution extraction with acetic acid;
adjusting the pH of the extract to 5 to 6 and removing the resulting precipitate;
separating a protein polysaccharide component by gel filtration; and
dialyzing the separated protein polysaccharide component and purifying the protein polysaccharide by desalinization,
to produce a (1→6)-β-D-glucan-containing product which is a protein polysaccharide of a (1→6)-β-D-glucan, which has a sugar content based on glucose of 43.3% as determined by the phenol sulfuric acid method and a protein content based on bovine albumin of 50.2% as determined by the Lowry method.

43. A method of inhibiting angiogenesis in a patient, the method comprising the steps of:
detecting undesired angiogenesis in the patient; and
administering to the patient a (1→6)-β-D-glucan-containing product which is a protein polysaccharide having the physicochemical properties (a) to (h) shown below,
(a) Elemental analysis:
C 41.87%
H 7.15%
N 6.88%
Sugar content 50.2%
Protein content 43.3%
(b) Molecular weight: 10,000 to 50,000
(c) Specific rotation:
[α]_{D}^{25≡}−23.0°
(c=1.0, 5% NaOH)
(d) Infrared absorption spectrum: Shown in FIG. 1.
(e) Nuclear magnetic resonance spectrum: Shown in FIG. 2.
(f) Solubility: Soluble in alkalis, and insoluble in organic solvents.
(g) Color reaction: Positive for phenol sulfuric acid and ninhydrin reaction.
(h) Amino acid composition (mol %):
Asp 10.7
Thr 5.2
Ser 5.3
Glu 11.1
Gly 9.3
Ala 11.9
Val 4.9
Met 1.1
Ile 3.3
Leu 10.8
Tyr 2.4
Phe 4.5
Lys 5.3
His 2.1
Arg 5.2
Pro 6.9
Total 100%.

44. A method in accordance with claim 43, wherein:
said patient has undesired angiogenesis induced by vascular endothelial growth factor; and
said protein polysaccharide is administered to the patient to inhibit angiogenesis induced by vascular endothelial growth factor.

45. A method in accordance with claim 43, wherein:
said patient has undesired growth of venous endothelial cells; and
said protein polysaccharide is administered to the patient to directly suppress the growth of venous endothelial cells.
